(19) United States
(12) Patent Application Publication
(10) Pub. No.: US 2024/0341791 A1
(43) Pub. Date: Oct. 17, 2024

(54) CLOT RETRIEVAL DEVICE FOR REMOVING HETEROGENEOUS CLOTS FROM A BLOOD VESSEL

(71) Applicant: Neuravi Limited, Galway (IE)

(72) Inventors: Anushree DWIVEDI, Galway (IE); Patrick CONNOLLY, Galway (IE)

(73) Assignee: Neuravi Limited, Galway (IE)

(21) Appl. No.: 18/754,867

(22) Filed: Jun. 26, 2024

Related U.S. Application Data
(63) Continuation of application No. 18/197,173, filed on May 15, 2023, now Pat. No. 12,048,446.
Application No. 18/197,173 is a division of application No. 16/852,104, filed on Apr. 17, 2020, now Pat. No. 11,717,308.

(51) Int. Cl. A61B 17/221; A61B 17/22

(52) U.S. Cl. CPC A61B 17/221; A61B 2017/22001

(57) ABSTRACT

A clot removal device can include a caged portion which can include a distal end; a proximal end; an inner cage having a network of inner struts; and an outer cage having a network of outer struts. The inner cage and the outer cage can include a delivery configuration within a microcatheter and a deployed configuration distal of the microcatheter operable to retrieve at least a portion of the clot. The device can include a distal pinching portion located proximate the distal end of the caged portion, and a proximal pinching portion located proximate the proximal end of the caged portion, each pinching portion can include at least one pinching cell can include a collapsed state and an expanded state distal of the microcatheter operable to tweeze at least a portion of the clot.

### CROSS-REFERENCE TO RELATED APPLICATIONS

The present application is a continuation application of U.S. patent application Ser. No. 18/197,173 filed May 15, 2023, which is a divisional application of U.S. patent application Ser. No. 16/852,104 filed Apr. 17, 2020, now U.S. Pat. No. 11,717,308 issued Aug. 8, 2023. The entire contents of which are hereby incorporated by reference.

### FIELD

The present disclosure generally relates to devices and methods for removing blockages from blood vessels during intravascular medical treatments.

### BACKGROUND

Clot retrieval devices are used in mechanical thrombectomy for endovascular intervention, often in cases where patients are suffering from conditions such as acute ischemic stroke (AIS), myocardial infarction (MI), and pulmonary embolism (PE). Acute obstructions may include clot, misplaced devices, migrated devices, large emboli and the like. Thromboembolism occurs when part or all of a thrombus breaks away from the blood vessel wall. This clot (now called an embolus) is then carried in the direction of blood flow. An ischemic stroke may result if the clot lodges in the cerebral vasculature. A pulmonary embolism may result if the clot originates in the venous system or in the right side of the heart and lodges in a pulmonary artery or branch thereof. Clots may also develop and block vessels locally without being released in the form of an embolus—this mechanism is common in the formation of coronary blockages. There are significant challenges associated with designing clot removal devices that can deliver high levels of performance. First, there are a number of access challenges that make it difficult to deliver devices. In cases where access involves navigating the aortic arch (such as coronary or cerebral blockages) the configuration of the arch in some patients makes it difficult to position a guide catheter. These difficult arch configurations are classified as either type 2 or type 3 aortic arches, with type 3 arches presenting the most difficulty.

The tortuousity challenge is even more severe in the arteries approaching the brain. For example, it is not unusual at the distal end of the internal carotid artery that the device will have to navigate a vessel segment with a 180° bend, a 90° bend and a 360° bend in quick succession over a few centimeters of vessel. In the case of pulmonary embolisms, access is through the venous system and then through the right atrium and ventricle of the heart. The right ventricular outflow tract and pulmonary arteries are delicate vessels that can easily be damaged by inflexible or high profile devices. For these reasons, it is desirable that the clot retrieval device be compatible with as low profile and flexible a guide catheter as possible.

Second, the vasculature in the area in which the clot may be lodged is often fragile and delicate. For example, neurovascular vessels are more fragile than similarly sized vessels in other parts of the body and are in a soft tissue bed. Excessive tensile forces applied on these vessels could result in perforations and hemorrhage. Pulmonary vessels are larger than those of the cerebral vasculature, but are also delicate in nature, particularly those more distal vessels.

Third, the clot may comprise any of a range of morphologies and consistencies. Long strands of softer clot material may tend to lodge at bifurcations or trifurcations, resulting in multiple vessels being simultaneously occluded over significant lengths. More mature and organized clot material is likely to be less compressible than softer fresher clot, and under the action of blood pressure it may distend the compliant vessel in which it is lodged. Furthermore, the inventors have discovered that the properties of the clot may be significantly changed by the action of the devices interacting with it. In particular, compression of a blood clot causes dehydration of the clot and results in a dramatic increase in both clot stiffness and coefficient of friction.

The challenges described above need to be overcome for any devices to provide a high level of success in removing clot and restoring flow. Existing devices do not adequately address these challenges, particularly those challenges associated with vessel trauma and clot properties.

### SUMMARY

It is an object of the present design to provide devices and methods to meet the above-stated needs. It is therefore desirable for a clot retrieval device to remove a clot from cerebral arteries in patients suffering AIS, from coronary native or graft vessels in patients suffering from MI, and from pulmonary arteries in patients suffering from PE and from other peripheral arterial and venous vessels in which the clot is causing an occlusion.

In some examples, the device includes pinch features along at the site of an occlusion (e.g., in the mid internal carotid artery (ICA)). The device can be configured to reperfuse a vessel and/or remove a clot that has a fibrin core. In some examples, the fibrin core can be in a mid-or distal-position in the clot surrounded by relatively soft thrombus.

In some examples, the device can be configured to remove a clot in the M1 bifurcation.

In some examples, the device can be configured to remove a clot in the M2 bifurcation.

In some examples, the device can include a caged portion which can include a distal end; a proximal end; an inner cage having a network of inner struts; and an outer cage having a network of outer struts. The inner cage and the outer cage can include a delivery configuration within a microcatheter and a deployed configuration distal of the microcatheter operable to retrieve at least a portion of the clot. The device can include a distal pinching portion located proximate the distal end of the caged portion, and a proximal pinching portion located proximate the proximal end of the caged portion, each pinching portion can include at least one pinching cell can include a collapsed state and an expanded state distal of the microcatheter operable to tweeze at least a portion of the clot.

In some examples, each pinching cell can include a plurality of strut members configured to actuate and pinch the clot between the plurality of strut members.

In some examples, the plurality of strut members can be positioned about a central strut member of the plurality of strut members, each strut member joined at common respective proximal and distal ends.

In some examples, each pinching cell can be operable to tweeze the clot on movement from the collapsed state to a clot pinching state of the expanded state until a portion of the clot can be compressed between the plurality of strut members.

In some examples, each pinching cell can include a ratio of diameters of each pinching cell between the collapsed state and the expanded state can be from approximately 1.5:1 to 4:1.

In some examples, each pinching cell can include a radiopaque marker disposed on the plurality of strut members.

In some examples, each pinching cell can include a pinching structure having a plurality of strut members and a central strut member of the plurality of strut members; a first collar having a first collar lumen; and a second collar having a second collar lumen; wherein the plurality of strut members and the central strut member connect the first collar to the second collar.

In some examples, the inner cage can be a plurality of pinching cells operable to tweeze at least a portion of the clot.

In some examples, each cell of the plurality of pinching cells can include a pinching structure having a plurality of strut members and a central strut member of the plurality of strut members; a first collar having a first collar lumen; and a second collar having a second collar lumen; wherein the plurality of strut members and the central strut member connect the first collar to the second collar.

In some examples, the plurality of pinching cells can include at least one radiopaque marker disposed on the pinching structure.

In some examples, each cell of the plurality of pinching cells can include the collapsed state and the expanded state distal of the microcatheter operable to tweeze at least a portion of the clot.

In some examples, the device can include an elongated member which can include a distal end connected to a proximal end of the proximal pinching portion, the elongated member operable to move the clot retrieval device in a distal or proximal direction.

In some examples, the network of struts can be connected to the network of inner struts.

In some examples, a method for removing a clot is disclosed. The method can include deploying a pinching portion of a clot retrieval device into an expanded state from a collapsed state within a blood vessel and proximate the clot. The clot retrieval device can include a caged portion, the caged portion can include a distal end, a delivery configuration within a microcatheter and a deployed configuration distal of the microcatheter operable to retrieve at least a portion of the clot. The pinching portion can be located proximate the distal end of the caged portion and can include the collapsed state and the expanded state distal of the microcatheter operable to pinch at least a portion of the clot. The method can include advancing a lumen of the microcatheter over the pinching portion such that the pinching portion at least partially collapses into the lumen of the microcatheter. The method can include pinching the pinching portion in contact with the portion of the clot on movement from the collapsed state to a clot pinching state of the expanded state until a portion of the clot can be compressed between the pinching portion and the microcatheter.

In some examples, the method can include determining that a portion of the clot is pinched; and withdrawing the microcatheter, the clot retrieval device, and the clot from the blood vessel while maintaining the clot in the clot pinching state of the pinching portion.

In some examples, the method can include determining that a portion of the clot is not pinched; deploying the caged portion of the clot retrieval device into the deployed configurations from the delivery configuration within the clot such that the caged portion can be operable to capture at least a portion of the clot; and retracting the microcatheter, the clot retrieval device, and the clot from the blood vessel while the clot remains embedded in the caged portion.

In some examples, the pinching portion can include a pinching structure having a plurality of strut members and a central strut member of the plurality of strut members; a first collar having a first collar lumen; and a second collar having a second collar lumen; wherein the plurality of strut members and the central strut member connect the first collar to the second collar.

In some examples, a method for removing a clot is disclosed. The method includes deploying a pinching portion of a clot retrieval device into an expanded state from a collapsed state within a blood vessel and proximate the clot, the pinching portion can be located proximate a distal end of a caged portion of the clot retrieval device, the pinching portion can include the collapsed state within a microcatheter and the expanded state distal of the microcatheter operable to pinch at least a portion of the clot. The method can include deploying the caged portion of the clot retrieval device into a deployed configuration from a delivery configuration within the blood vessel and proximate the clot, The caged portion can include the delivery configuration within the microcatheter and the deployed configuration distal of the microcatheter operable to retrieve at least a portion of the clot. The method can include advancing a lumen of the microcatheter over the pinching portion such that the pinching portion at least partially collapses into the lumen of the microcatheter; pinching the pinching portion in contact with the portion of the clot on movement from the collapsed state to a clot pinching state of the expanded state until a portion of the clot can be compressed between the pinching portion and the microcatheter. The method can include retracting the microcatheter, the clot retrieval device, and the clot from the blood vessel while the clot is pinched by the pinching portion.

In some examples, the device can include a proximal pinching portion located proximate the proximal end of the caged portion. The proximal pinching portion can include a proximal end; and an elongated member can include a distal end connected to the proximal end of the pinching portion. The elongated member can be operable to move the clot retrieval device in a distal direction or proximal direction.

In some examples, the pinching portion can include a pinching structure having a plurality of strut members and a central strut member of the plurality of strut members; a first collar having a first collar lumen; and a second collar can include a second collar lumen; wherein the plurality of strut members and the central strut member connect the first collar to the second collar.

Other aspects and features of the present disclosure will become apparent to those of ordinary skill in the art, upon reviewing the following detailed description in conjunction with the accompanying figures.

### BRIEF DESCRIPTION OF THE DRAWINGS

The above and further aspects of this disclosure are further discussed with the following description of the accompanying drawings, in which like numerals indicate like structural elements and features in various figures. The drawings are not necessarily to scale, emphasis instead being placed upon illustrating principles of the disclosure. The figures depict one or more implementations of the inventive devices, by way of example only, not by way of limitation. It is expected that those of skill in the art can conceive of and combining elements from multiple figures to better suit the needs of the user.

FIG. 1 illustrates an example clot removal device according to aspects of the present disclosure.

FIG. 2 depicts a close-up view of an example pinch section according to aspects of the present disclosure.

FIG. 3 illustrates an example clot removal device in a collapsed configuration according to aspects of the present disclosure.

FIG. 4 is a flowchart that depicts a method for an example clot removal device in a collapsed configuration according to aspects of the present disclosure.

FIG. 5 depicts an example clot removal device according to aspects of the present disclosure.

FIG. 6A illustrates example pinching cells. FIG. 6B illustrates example pinching cells. FIG. 6C illustrates example pinching cells. FIG. 6D illustrates example pinching cells.

FIG. 7A illustrates pinching cells in an expanded state. FIG. 7B illustrates pinching cells in a collapsed state. FIG. 7C illustrates pinching cells in a pinched state of the expanded state.

FIG. 8 illustrates an example clot removal device according to aspects of the present disclosure.

### DETAILED DESCRIPTION

Specific examples of the present disclosure are now described in detail with reference to the Figures, where identical reference numbers indicate elements which are functionally similar or identical. The examples address many of the deficiencies associated with traditional catheters, such as inefficient clot removal and inaccurate deployment of catheters to a target site.

Accessing the various vessels within the vasculature, whether they are coronary, pulmonary, or cerebral, involves well-known procedural steps and the use of a number of conventional, commercially-available accessory products. These products, such as angiographic materials and guidewires are widely used in laboratory and medical procedures. When these products are employed in conjunction with the system and methods of this disclosure in the description below, their function and exact constitution are not described in detail.

The following detailed description is merely exemplary in nature and is not intended to limit the disclosure or the application and uses of the disclosure. Although the description of the disclosure is in many cases in the context of treatment of intracranial arteries, the disclosure may also be used in other body passageways as previously described.

It will be apparent from the foregoing description that, while particular embodiments of the present disclosure have been illustrated and described, various modifications can be made without departing from the spirit and scope of the disclosure. For example, while the embodiments described herein refer to particular features, the disclosure includes embodiments having different combinations of features. The disclosure also includes embodiments that do not include all of the specific features described. Specific embodiments of the present disclosure are now described in detail with reference to the figures, wherein identical reference numbers indicate identical or functionality similar elements. The terms “distal” or “proximal” are used in the following description with respect to a position or direction relative to the treating physician. “Distal” or “distally” are a position distant from or in a direction away from the physician. “Proximal” or “proximally” or “proximate” are a position near or in a direction toward the physician.

Accessing cerebral, coronary, and pulmonary vessels involves the use of a number of commercially available products and conventional procedural steps. Access products such as guidewires, guide catheters, angiographic catheters and microcatheters are described elsewhere and are regularly used in catheter lab procedures. It is assumed in the descriptions below that these products and methods are employed in conjunction with the device and methods of this disclosure and do not need to be described in detail.

The following detailed description is merely exemplary in nature and is not intended to limit the disclosure or the application and uses of the disclosure. Although the description of the disclosure is in many cases in the context of treatment of intracranial arteries, the disclosure may also be used in other body passageways as previously described.

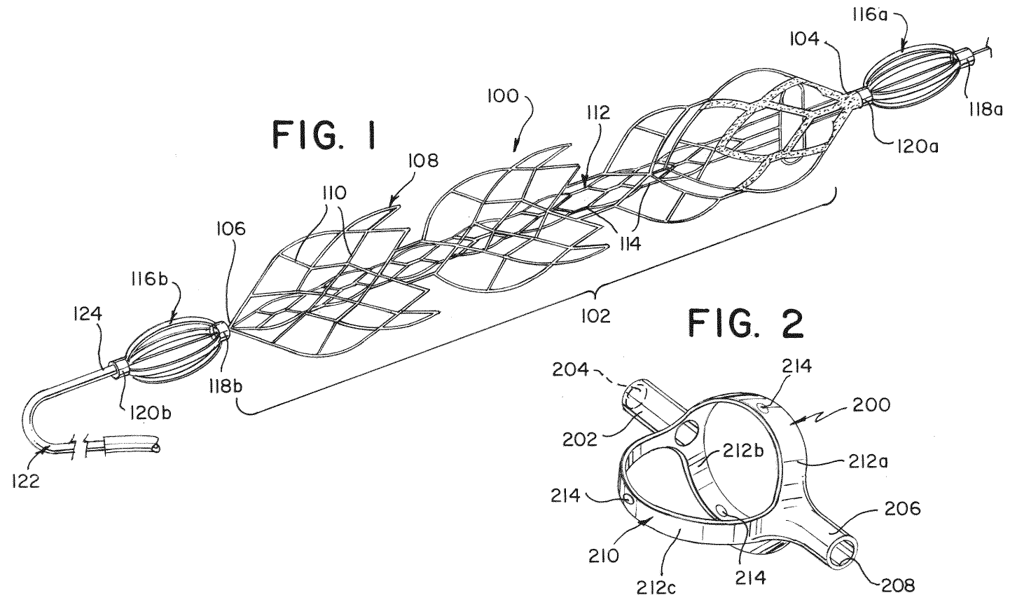

A common theme across many of the disclosed designs is a multi-layer construction in which the device in certain instances can include an outer cage within which, at times, can include an inner cage, both cages being directly or indirectly connected to an elongate member. Turning to FIG. 1, one example device 100 according to this disclosure is illustrated. Device 100 can include a caged portion 102 having a distal end 104 and a proximal end 106. The caged portion 102 can include an outer cage 108 made of a network of outer struts 110. The caged portion 102 can include an inner cage 112 made of a network of inner struts 114. Device 100 can include a distal pinching portion 116a positioned distal of the caged portion 102 and having a distal end 118a and a proximal end 120a. The proximal end 120a of the distal pinching portion 116a can be attached at the distal end 104 of the caged portion 102. Device 100 can include a proximal pinching portion 116b positioned proximal of the caged portion 102 and having a distal end 118b and a proximal end 120b. The distal end 118b of the proximal pinching portion 116b can be attached at the proximal end 106 of the caged portion 102. In some examples, the one or more pinching portions 116a, 116b can be pinching cells operable to pinch, grip, or tweeze a clot, as will be discussed in detail in FIG. 2. As discussed herein, the term “tweeze” or “tweezing” is intended to refer to the sheathing of the pinching cells that causes respective struts to come together and tweeze or grip at least a portion of a clot. In this respect, while the numbers of struts in a respective cell need not be limited, at least two strut surfaces must be included so as to tweeze corresponding clot material.

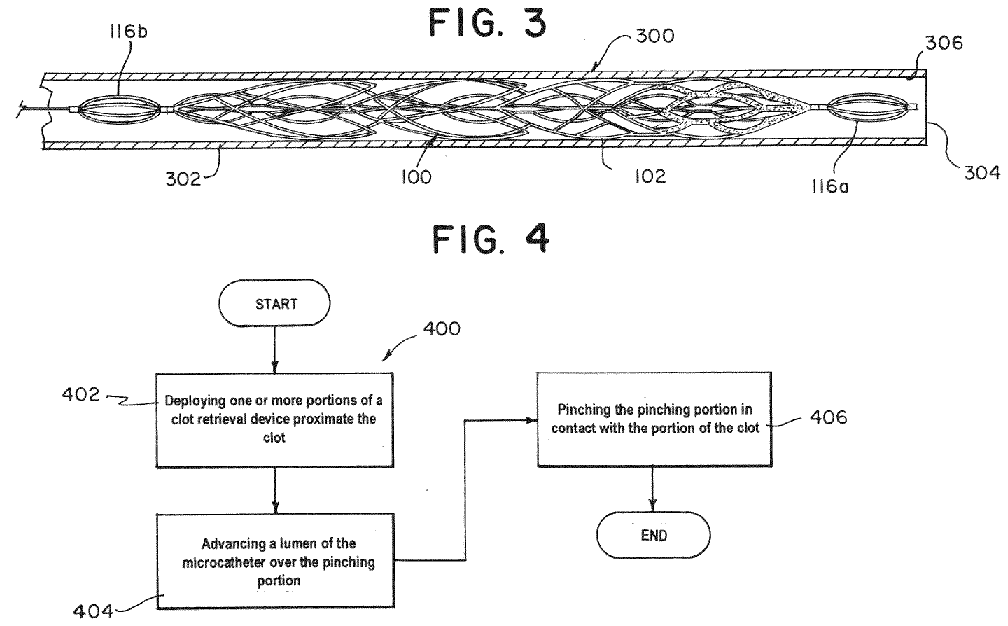

Device 100 can also include an elongated member 122 having a distal end 124. The distal end 124 of the elongated member 122 can be attached to the proximal end 120b of the proximal pinching portion 116b. Additionally or alternatively, the distal end 124 of the elongated member 122 can be attached to the caged portion 102. Device 100 can include a delivery configuration within a lumen of a microcatheter, as discussed in FIG. 3, and a deployed configuration distal of the microcatheter, as shown.

The elongated member 122 can be a tapered wire shaft, and may be made of stainless steel, MP35N, Nitinol or other material of a suitably high modulus and tensile strength. The caged portion 102 and the pinching portions 116a, 116b are desirably made from a material capable of recovering its shape automatically once released from a highly strained delivery configuration. A superelastic material such as Nitinol or an alloy of similar properties is particularly suitable. The material could be in many forms such as wire or strip or sheet or tube. A particularly suitable manufacturing process is to laser cut a Nitinol tube and then heat set and electropolish the resultant structure to create a framework of struts and connecting elements. This framework can be any of a huge range of shapes as disclosed herein and may be rendered visible under fluoroscopy through the addition of alloying elements (e.g., Platinum) or through a variety of other coatings or marker bands.

FIG. 2 depicts a close-up view of an example pinching cell 200. Pinching cells 200 can be configured to embed and/or engage with and grip the clot to retain it securely for retraction. It is understood that each of the herein described pinching cells can be used interchangeably with clot retrieval devices as needed or required. Pinching cell 200 can include a first collar 202, a first lumen 204, a second collar 206, and a second lumen 208 between which a pinching structure 210 is positioned (e.g., between the first and second collar). The pinching structure 210 can include strut members 212a, 212b, and 212c. One or more of strut members 212a, 212b, and 212c can be configured as bowed or otherwise including tensioned flex so as to be capable of embedding in a clot and then being actuated to grip and/or pinch the clot during use. The terms “bowed” is intended to refer to a strut that is generally a shape of an arc, while “tension flex” is intended to refer to a strut that has been placed in tension and plastically deformed into a desired shape. Pinching cell 200 can include radiopaque markers 214 disposed on the one or more strut members.

In some examples, pinching cell 200 can be actuated into the pinched state by being unsheathed from a sheath (e.g., a microcatheter), by being pulled, or actuated by one or more pull members, or by delivering an electric current to one or more of strut members 212a, 212b, and 212c to cause at least a first portion of the one or more of strut members 212a, 212b, and 212c to change from a collapsed state to pinch state. The pinching cell 200 can be configured to embed and grip, pinch, and/or “tweeze” the clot, as shown and described more particularly in FIGS. 7A-C. One or more of strut members 212a, 212b, and 212c can also have one or more radiopaque bands to indicate to the user when the pinching cell 200 is pinched, since the distance between struts is decreased when the pinching cell 200 is in a pinched state of the expanded state.

The diameter of pinching cell 200 can range between approximately 2-10 millimeters, as needed or required. One preferred diameter can be approximately 2.25 millimeters. In some examples, pinching cells 200 can be small enough to fit in a 0.021 or 0.018 inch ID microcatheter. The pinching cell 200 can be constructed from a superelastic material such as Nitinol or an alloy of similar properties. The material could be in many forms such as wire or strip or sheet or tube. A particularly suitable manufacturing process is to laser cut a Nitinol tube and then heat set and electropolish the resultant structure to create a framework of struts. This framework can be any of huge range of shapes as disclosed herein and may be rendered visible under fluoroscopy through the addition of alloying elements (e.g., platinum) or through a variety of other coatings or marker bands.

Turning to FIG. 3, device 100 is shown in a delivery configuration collapsed within the delivery system 300. In particular, device 100 is in a delivery configuration within a lumen 306 of the microcatheter 302. The microcatheter 302 can have a distal end 304. Further, the pinching cells 200, can be in a collapsed state, as discussed in detail in FIGS. 7A-C.

FIG. 4 is a flow diagram illustrating a method of removing a clot from a blood vessel of a patient, according to aspects of the present disclosure. The method steps in FIG. 4 can be implemented by any of the example means described herein or by similar means, as will be appreciated. Referring to method 400 as outlined in FIG. 4, in step 402, deploying a pinching portion of a clot retrieval device into an expanded state from a collapsed state within a blood vessel and proximate the clot, the clot retrieval device can include a caged portion. The caged portion having a distal end, a delivery configuration within a microcatheter and a deployed configuration distal of the microcatheter operable to retrieve at least a portion of the clot. The pinching portion can be located proximate the distal end of the caged portion, and can include the collapsed state and the expanded state distal of the microcatheter operable to pinch at least a portion of the clot. Additionally or alternatively, step 402 can include deploying the caged portion of the clot retrieval device into the deployed configurations from the delivery configuration within the clot such that the caged portion is operable to capture at least a portion of the clot. In step 404, advancing a lumen of the microcatheter over the pinching portion such that the pinching portion at least partially collapses into the lumen of the microcatheter.

In step 406, pinching the pinching portion in contact with the portion of the clot on movement from the collapsed state to a clot pinching state of the expanded state until a portion of the clot is compressed between the pinching portion and the microcatheter. The method can further include determining whether the clot is pinched. Determining that the clot is pinched, the method can include withdrawing the microcatheter, the clot retrieval device, and the clot from the blood vessel while maintaining the clot in the clot pinching state of the pinching portion. Determining that the clot is not pinched, the method can include deploying the caged portion of the clot retrieval device into the deployed configurations from the delivery configuration within the clot such that the caged portion is operable to capture at least a portion of the clot; and retracting the microcatheter, the clot retrieval device, and the clot from the blood vessel while the clot remains entangled in the caged portion. Method 400 can end after step 406. In other embodiments, additional steps according to the examples described above can be performed.

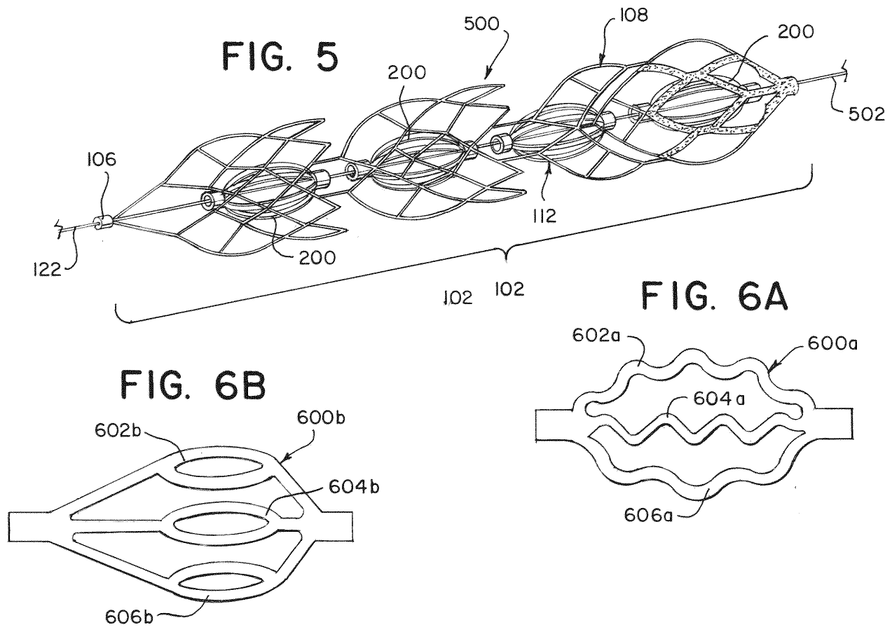

FIG. 5 illustrates an example clot removal device. Device 500 can include an inner cage 112 of the caged portion 102 comprised of one or more pinching cells 200. Cells 200 can be sequentially arranged end-to-end along a common axis of shaft 502. Cells 200 can be in contact with each other (e.g., distal end of a first cell 200 touching a proximal end of a second cell 200, and so forth). In other examples, cells 200 can each be separated a predetermined distance and/or positioned in a one-to-one ratio with caged portions of device 500. In some examples, more than one cell 200 can be included per caged portion. The inner cage 112 can be within an outer cage 108. The proximal end 106 of the caged portion 102 can be operable to attach to an elongated member 122 as discussed in detail above.

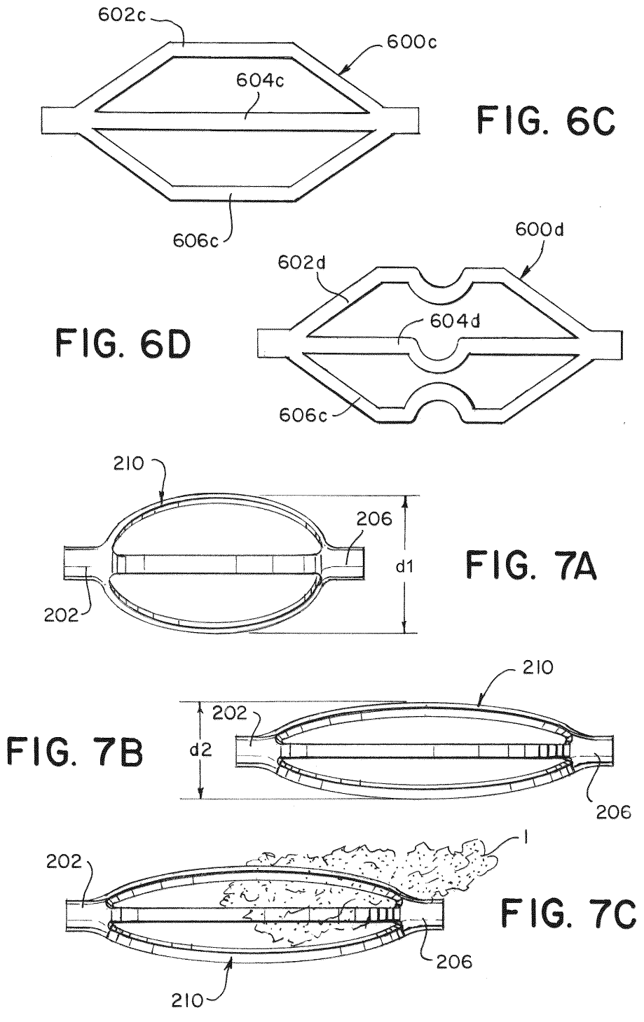

FIG. 6A depicts a close-up view of another example pinching cell 600a with strut members 602a, 604a, and 606a now shown with undulating edges. These undulations can be formed by being heat-set, crimped, or otherwise formed as needed or required. FIG. 6B depicts a close-up view of another example pinching cell 600b with strut members 602b, 604b, and 606b each including one or more eyelets. FIG. 6C depicts a close-up view of another example pinching cell 600c with strut members 602c, 604c, and 606c now shown with relatively straight, non-curved strut members. FIG. 6D depicts a close-up view of another example pinching cell 600d with strut members 602d, 604d, and 606d each including one or more notches or indentation. These notches or indentations can be formed by being heat-set, crimped, or otherwise formed as needed or required.

FIGS. 7A-C illustrate pinching cell states. An example expanded state of the pinching cell 200 is depicted in FIG. 7A. The pinching structure 210 has an expanded diameter D1 which can be realized distal of the distal end 304 of the microcatheter 302. An example collapsed state of the pinching cell 200 is depicted in FIG. 7B. The pinching structure 210 has a collapsed diameter D2 which can be realized within the lumen 306 of the microcatheter 302. An example pinched state of the expanded state of the pinching cell 200 is depicted in FIG. 7C, where the pinching structure 210 is pinching clot I. The pinching structure 210 in the pinched state of the expanded state can have a diameter less than the expanded diameter D1, but greater than the collapsed diameter D2. A ratio of diameters can be calculated by dividing the expanded diameter D1 by the collapsed diameter D2. Alternatively, a ratio can be computed by dividing the collapsed diameter D1 by the expanded diameter D2.

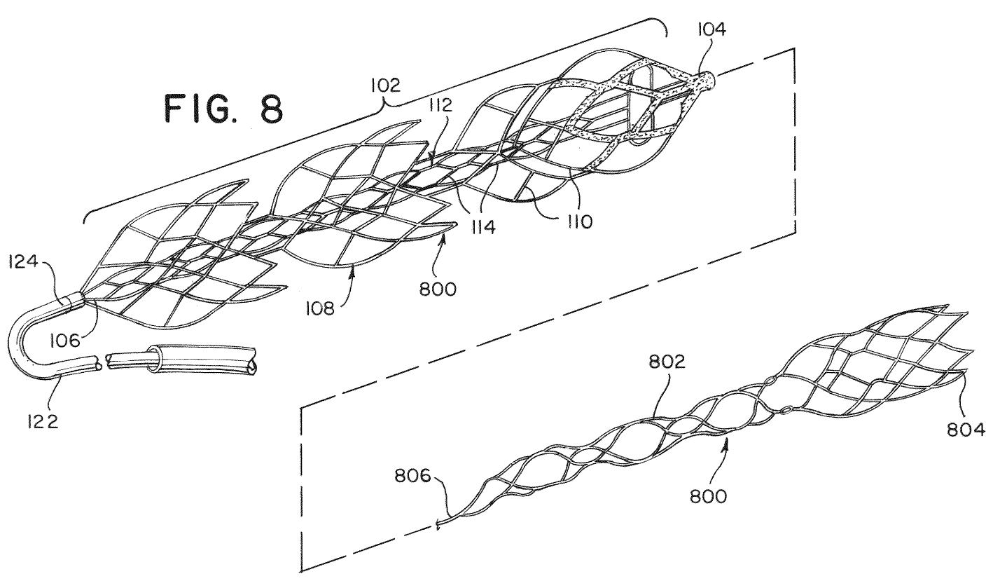

FIG. 8 illustrates an example clot removal device. Device 800 can include a caged portion 102, an elongated member 122 having a distal end 124, wherein the distal end 124 of the elongated member 122 can connect to a proximal end 106 of the caged portion 102. The elongated member 122 is operable to move at least the caged portion 102 in a distal or proximal direction upon moving the elongated member 122, whereby such features of device 800 can be understood as including features, features, and designs described in U.S. Pat. Nos. 8,777,976; 8,852,205; 9,402,707; 9,445,829; and 9,642,639, each of which are incorporated by reference in their entirety as if set forth verbatim herein.

The device 800 of FIG. 8 can also include a pinching portion 802 located adjacent a distal end 104 of the caged portion 102. The pinching portion 802, which can be elongated in certain examples, has a distal end 804 and a proximal end 806. The proximal end 806 of the elongated pinching portion 802 is connected to the distal end 104 of the caged portion 802. The elongated pinching portion 802 can be a network of struts in a tubular shape and operable to grip a clot. Similar to the pinching cell 200, the pinching portion 802 can have an expanded state, a collapsed state and a clot pinching state of the expanded state, whereby caged portion 802 can include a variety of shapes and designs configured for pinching fibrin rich clots, including those described in U.S. Pat. Nos. 10,292,723; 10,363,054; U.S. application Ser. Nos. 15/359,943; 16/021,505; and 16/330,703, each of which are incorporated by reference in their entirety as if set forth verbatim herein.

The disclosure is not limited to the examples described, which can be varied in construction and detail. The terms “distal” and “proximal” are used throughout the preceding description and are meant to refer to a positions and directions relative to a treating physician. As such, “distal” or distally” refer to a position distant to or a direction away from the physician. Similarly, “proximal” or “proximally” refer to a position near to or a direction towards the physician.

In describing examples, terminology is resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method can be performed in a different order than those described herein without departing from the scope of the disclosed technology. Similarly, it is also to be understood that the mention of one or more components in a device or system does not preclude the presence of additional components or intervening components between those components expressly identified.

As discussed herein, a “patient” or “subject” can be a human or any animal. It should be appreciated that an animal can be a variety of any applicable type, including, but not limited to, mammal, veterinarian animal, livestock animal or pet-type animal, etc. As an example, the animal can be a laboratory animal specifically selected to have certain characteristics similar to a human (e.g., rat, dog, pig, monkey, or the like).

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g., “about 90%” may refer to the range of values from 71% to 99%. Ranges can be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” or “having” is meant that at least the named compound, clement, particle, or method step is present in the composition or article or method, but does not exclude the presence of other compounds, materials, particles, method steps, even if the other such compounds, material, particles, method steps have the same function as what is named.

It must also be noted that, as used in the specification and the appended claims, the singular forms “a,” “an,” and “the” include plural referents unless the context clearly dictates otherwise.

The descriptions contained herein are examples of the disclosure and are not intended in any way to limit the scope of the disclosure. While particular examples of the present disclosure are described, various modifications to devices and methods can be made without departing from the scope and spirit of the disclosure. For example, while the examples described herein refer to particular components, the disclosure includes other examples utilizing various combinations of components to achieve a described functionality, utilizing alternative materials to achieve a described functionality, combining components from the various examples, combining components from the various example with known components, etc. The disclosure contemplates substitutions of component parts illustrated herein with other well-known and commercially-available products. To those having ordinary skill in the art to which this disclosure relates, these modifications are often apparent and are intended to be within the scope of the claims which follow.

What is claimed is:

1. A clot retrieval device for retrieving a clot from a blood vessel, comprising:
a caged portion, the caged portion comprising a distal end, a proximal end, a delivery configuration within a microcatheter, and a deployed configuration distal of a microcatheter operable to retrieve a first portion of the clot; and
a pinching portion positioned distal of the distal end of the caged portion, the pinching portion comprising a collapsed state within the microcatheter and an expanded state distal of the microcatheter operable to pinch a second portion of the clot.

2. The clot retrieval device of claim 1, wherein the pinching portion is elongated.

3. The clot retrieval device of claim 1, wherein the pinching portion comprises a network of struts in a tubular shape.

4. The clot retrieval device of claim 3, wherein the pinching portion further comprises a proximal portion and a distal portion.

5. The clot retrieval device of claim 4, wherein a diameter of the proximal portion is smaller than a diameter of the distal portion.

6. The clot retrieval device of claim 1, wherein the caged portion further comprises:
an inner cage comprising a network of inner struts; and
an outer cage comprising a network of struts.

7. The clot retrieval device of claim 6, wherein the inner cage is a plurality of pinching cells operable to tweeze the second portion of the clot.

8. The clot retrieval device of claim 7, wherein each cell of the plurality of pinching cells further comprises:
a pinching structure comprising a plurality of strut members and a central strut member of the plurality of strut members;
a first collar comprising a first collar lumen; and
a second collar comprising a second collar lumen; and
wherein the plurality of strut members and the central strut member connect the first collar to the second collar.

9. The clot retrieval device of claim 1, wherein the clot retrieval device further comprises:
a proximal pinching portion located proximate the proximal end of the caged portion, the proximal pinching portion comprising a proximal end; and
an elongated member comprising a distal end connected to the proximal end of the proximal pinching portion, the elongated member operable to move the clot retrieval device in a distal direction or proximal direction.

10. A method for retrieving a clot, the method comprising the steps of:
deploying a distal pinching portion of a clot retrieval device into an expanded state from a collapsed state within a blood vessel and proximate the clot, the distal pinching portion positioned distal of a distal end of a caged portion of the clot retrieval device, the distal pinching portion comprising the collapsed state within a microcatheter and the expanded state distal of the microcatheter operable to pinch a first portion of the clot;
deploying the caged portion of the clot retrieval device into a deployed configuration from a delivery configuration within the blood vessel and proximate a second portion of the clot, the caged portion comprising the delivery configuration within the microcatheter and the deployed configuration distal of the microcatheter operable to retrieve at least the second portion of the clot;
advancing a lumen of the microcatheter over the pinching portion such that the distal pinching portion at least partially collapses into the lumen of the microcatheter;
pinching the distal pinching portion in contact with the first portion of the clot on movement from the collapsed state to a clot pinching state of the expanded state until the first portion of the clot is compressed between the distal pinching portion and the microcatheter; and
retracting the microcatheter, the clot retrieval device, and the first portion of the clot from the blood vessel while the first portion of the clot is pinched by the distal pinching portion.

11. The method of claim 10, wherein the clot retrieval device further comprises:
a proximal pinching portion located proximal the proximal end of the caged portion, the proximal pinching portion comprising a proximal end; and
an elongated member comprising a distal end connected to the proximal end of the proximal pinching portion, the elongated member operable to move the clot retrieval device in a distal direction or proximal direction.

12. The method of claim 10, wherein the distal pinching portion further comprises a network of struts in a tubular shape, wherein a proximal end of the distal pinching portion has a diameter less than a distal end of the distal pinching portion.

13. The method of claim 10, the method further comprising the steps of:
determining that the first portion of the clot is pinched; and
withdrawing the microcatheter, the clot retrieval device, and the first portion of the clot from the blood vessel while maintaining the first portion of the clot in the clot pinching state of the expanded state.

14. The method of claim 10, the method further comprising the steps of:
determining that the second portion of the clot is not pinched;
deploying the caged portion of the clot retrieval device into the deployed configuration from the delivery configuration within the second portion of the clot such that the caged portion is operable to capture the second portion of the clot; and
retracting the microcatheter, the clot retrieval device, and the second portion of the clot from the blood vessel while the second portion of the clot remains entangled in the caged portion.

15. The method of claim 12, wherein the network of struts is operable to tweeze the first portion of the clot on movement from the collapsed state to a clot pinching state of the expanded state until the first portion of the clot is compressed between the network of struts.

16. The method of claim 10, wherein the caged portion further comprises:
an inner cage comprising a network of inner struts; and
an outer cage comprising a network of struts.

17. The method of claim 16, wherein the inner cage is a plurality of pinching cells operable to tweeze the first portion of the clot.

18. The method of claim 17, wherein each cell of the plurality of pinching cells further comprises:
a pinching structure comprising a plurality of strut members and a central strut member of the plurality of strut members;
a first collar comprising a first collar lumen; and
a second collar comprising a second collar lumen; and
wherein the plurality of strut members and the central strut member connect the first collar to the second collar.

19. The method of claim 10, wherein the clot retrieval device further comprises:
a proximal pinching portion located proximate the proximal end of the caged portion, the proximal pinching portion comprising a proximal end; and
an elongated member comprising a distal end connected to the proximal end of the proximal pinching portion, the elongated member operable to move the clot retrieval device in a distal direction or proximal direction.
